(19) United States
(12) Patent Application Publication
(10) Pub. No.: US 2013/0066403 A1
(43) Pub. Date: Mar. 14, 2013

(54) DEVICE FOR DERMATOLOGICAL TREATMENT USING A LASER BEAM

(71) Applicants: Sylvain Giraud, Gardanne (FR); Alban Gosse, Mimet (FR); Alain Cornil, Aix En Provence (FR); Patrick Peronne, Aix En Provence (FR)

(72) Inventors: Sylvain Giraud, Gardanne (FR); Alban Gosse, Mimet (FR); Alain Cornil, Aix En Provence (FR); Patrick Peronne, Aix En Provence (FR)

(73) Assignee: EKKYO, Aix en Provence (FR)

(21) Appl. No.: 13/516,537

(22) PCT Filed: Dec. 29, 2010
(86) PCT No.: PCT/IB2010/003358

§ 371 Date: Nov. 27, 2012

(30) Foreign Application Priority Data
Dec. 29, 2009 (FR) 09 06375

(51) Int. Cl. A61N 5/067

(52) U.S. Cl. 607/89

(57) ABSTRACT

A dermatological treatment device using a light beam, includes: a laser source (1) suitable for directing a laser beam onto at least one zone (2) of the area of skin to be treated, a contactless measurement unit sensitive to radiation according to temperature, of the temperature of a skin area corresponding to the area of skin being treated, and a unit for controlling (13) the laser source via the measurement unit, the device being characterised by the fact that the contactless measurement unit includes an infrared sensor (4) and an objective lens (20) suitable for focusing the field of view (5) of the infrared sensor such that the area of skin contained within the field of view (5) is fully included in the area of skin (2) being treated by the device.

### FIELD OF THE INVENTION

This invention concerns a device for dermatological treatment using a laser beam, preferably a laser beam having a wavelength from from 1.1 to 1.6 μm such as 1.21 μm

### PRIOR ART

It is known via document WO 2009/071592, that a device exists for perioperative and post-operative treatment of surgical wounds. This device uses a laser source with a wavelength from 0.6 μm to 2.5 μm, the beam of which is shaped such that, when the laser radiates the skin, energy distribution over the area to be treated is homogenous. More specifically, the beam is shaped in the form of a rectangle in order to match the geometry of an incision, the incision then being treated in several sections of a size equalling the length of the rectangle formed by the laser beam.

The difficulty with this device lies in the fact that the power of the laser and the length of exposure for each section must be determined in order to heat the edges of the incisions to a temperature that must be precisely between 45° C. and 55° C. (a temperature lower than 45° C. being ineffective and a temperature higher than 60° C. causing burning). Within this range, heating generates thermal stress in the dermis which is expressed by the production of specific proteins (HSP: heat shock proteins) intervening in the natural healing mechanism. Heating therefore promotes more rapid and better organised regeneration of the tissue that has been subject to incision. Finally, healing is thus facilitated and is, in the end, less visible.

In order to limit the risk of exceeding 60° C. and burning the patient, the laser can be combined with a safety strip of the type described in document WO 2008/107563. This strip ensures users' safety because the laser can only be activated when close to it (for example less than 5 mm). In order to modulate the firing power of the laser, there are in addition different types of strip according to the patient's skin type. In fact, skin types can be grouped into categories (six according to the Fitz-Patrick test: Phototype I for very white skin to Phototype VI for very black skin). The practitioner therefore chooses the strip associated with the patient's phototype, the strip permitting the laser device to adjust its treatment parameters (power, time) to the patient's phototype.

The inventors shown that the use of a laser source having a wavelength of 1210 nm instead of 810 nm enable to obtain a heating with nearly no influence of the skin type.

However, the patient's phototype is not the only parameter that influences the temperature reached (final temperature) during treatment by the laser device; the following variables also have an influence:
The temperature of the zone before treatment by the laser (initial temperature).
The presence of blood in the incision, in the edges of the incision and/or under the incision exerts a strong influence because blood heats more rapidly than lightly pigmented skin. Of course, this factor depends significantly on the type of operation and the practitioner's surgical technique. The inventors demonstrated that at a wavelength of 1210 nm, pure blood and a bloodless skin surface are subject to more or less the same heating. The quantity of blood present in the incision will exert a weak influence on the heating of the area irradiated by the laser and will therefore have a weak influence on the treatment of the scar.
The vascularisation of the irradiated tissue. The more tissue is irrigated by heated blood, the more it will heat rapidly, still due to the fact that blood absorbs heat more quickly as compared with other components of the skin.
The thickness of the skin that has been subject to incision. Presently, the inventors observed that the heating obtained with a laser source having a wavelength of 1210 nm is homogeneous to a depth of at least 2 mm.

These parameters are difficult to measure during routine use of the device and may be extremely variable depending on the patient and the practitioners' techniques. As a consequence, it is difficult to predict the temperature reached during treatment using the laser device accurately and the risk cannot be excluded of exceeding 60° C. (burning) or of the temperature remaining below 45° C. (ineffective treatment).

In order to mitigate similar difficulties, the prior art has envisaged controlling the temperature rise in the skin by placing the laser device under the control of an infrared sensor (pyrometer) that monitors the temperature of the patient's skin in real-time, and if necessary, modulates the power of the laser beam on the skin. A schematic diagram of such devices is envisaged in particular in patent U.S. Pat. No. 5,409,481 and in patent application US 2007/0179484.

It is in fact known that contactless measurement of the temperature of any body can be achieved by measuring the infrared radiation emitted by this body. In fact, according to Planck's law, every body emits infrared radiation, the wavelength and power of which are linked to temperature. For example, a body. at 300° K (23° C.) essentially emits infrared radiation within a wavelength range from 6 μm to 10 μm.

The power E radiated per unit of area for a body with a temperature T_{obj} is given by:

E=ε*ℑ*T_{obj}⁴

where ε is the emissivity of the body under consideration and where σ is the Stefan-Boltzmann constant.

The wavelength of the laser source used by dermatological treatment devices is within the range 0.6 μm to 2.5 μm. The temperatures to be measured are lower than 70°, corresponding to heat radiation within the range 6 μm to 10 μm. As the two ranges do not overlap, temperature measurement in the presence of laser radiation is therefore possible.

The creation of such a device does however present difficulties.

In the first place, the zone heated by the device's laser beam is generally small in size because a great amount of energy per unit of area is required to heat the zone to be treated. FIG. 1 illustrates this problem.

This figure shows a light source (1) irradiating and therefore heating a zone (2) of the skin (3) to be treated. The shape of the zone (2) may be, for example, circular or rectangular depending on the desired therapeutic application. The area S₁ of zone (2) is generally between 0.1 cm² and 2 cm².

This figure also shows the infrared sensor (4) intended to measure the temperature of zone (1). Its field of view on the skin (3) is zone (5). The fields of view of infrared thermal sensors are generally wide, that is a cone of vision, the total typical angle of which is 50° (varying from 20° to 70° depending on the type). By placing such a sensor 30 mm from the zone irradiated by the light beam, the area S₂ of zone (5) seen by the infrared sensor equals:

$${S}_{2} = 3 * tan ( \frac{{50}^{°}}{2} ) = 6.14 {cm}^{2}$$

therefore significantly greater than the area S₂ of zone (5).

The result of this is

T°₁>T°ₚ>T°₂

where

T°₁ is the temperature of zone (2) irradiated by the laser beam,

T°₂ is the temperature of the skin (3) that is not irradiated by the laser beam,

T°ₚ is the temperature measured by the infrared sensor.

FIG. (11) shows an example of the differences between temperatures T°₁ and T°ₚ in this context.

The temperature measured is therefore extremely imprecise because it is situated between the temperature of the irradiated zone and that of non-irradiated zone.

In order to solve this problem, moving the infrared sensor closer to the zone to be treated can be envisaged. However an additional difficulty then appears, this difficulty residing in the fact that the skin is a medium that gives rise to strong diffusion of light rays.

The result of this is that before being absorbed, photons may follow complex routes within the skin. As shown in FIG. (2), some (6) of these photons are caused to “come out of” skin once more, seeming to have been emitted by the latter. In practice, about 10% of the light power incident on the patient is thus diffused and re-emitted outside the skin. This diffusion disturbs the operation of the infrared heat sensor in two manners.
The photons (6) resulting from the diffusion of the light beam (wavelength 0.6 μm to 2.5 μm) and the photons radiated by the skin due to its temperature (wavelength 6 μm to 10 μm) merge and add to each other. The infrared sensors are generally equipped with a wavelength sensitive selective filter in order to suppress this type of disturbance. However in this case, the power per unit of area of the diffusion of the light beam is from 10 to 15 times greater than the power per unit of area radiated by a body at a temperature of 45° C. The filter, even if it is effective, cannot be sufficient. Temperature measurements are therefore increasingly false as the power of the light beam increases. Compensating for this disturbance via calculation implies finding out the proportion of the power of the light beam that is diffused, a value which varies from one person's skin to another (between white skin and black skin for example).
The photons resulting from the diffusion of the light beam have a secondary effect of warming all the elements that are close to the zone undergoing treatment, in particular the temperature sensor. Thus, the temperature measurement determined by the sensor is calculated based on two items of information: the electrical signal returned by the sensor, and measurement of the temperature of the sensor itself. A temperature probe is therefore placed as close as possible to the infrared thermal sensor. Rapid heating such as that created by the diffusion of the light beam creates a transient heat balance between the infrared sensor and the temperature probe. The data from the temperature probe is thus falsified, as is the temperature calculated.

FIG. 12 shows an example of the differences between temperatures T° 1 and T° p in this context.

In the light of these additional difficulties linked to the distance away from, or proximity to the zone to be treated, none of the laser devices in the prior art have in practice been combined with an infrared sensor in the light of the significant inaccuracy of the measurements.

### SUMMARY OF THE INVENTION

The invention aims to mitigate these drawbacks and its purpose is to supply a dermatological treatment device using a light beam that ensures the effectiveness of the treatment, while eliminating risks of burning using an infrared sensor.

To this end, the first purpose of the invention is a dermatological treatment device using a light beam comprising:
a light source suitable for directing a light beam onto at least a zone of the surface of the skin to be treated.
a means of contactless measurement sensitive to radiation according to temperature, of the temperature of a skin surface corresponding to the skin zone treated, and
a means of controlling the aforesaid light source via the aforesaid means of measurement.

The aforesaid device being characterised in that the aforesaid means of contactless measurement comprises an infrared sensor and an objective lens suitable for focusing the field of view of the aforesaid infrared sensor such that the skin surface contained within the aforesaid field of view is wholly included in the area of skin treated by the aforesaid device.

The purpose of the invention is also a system for dermatological treatment using a light beam, the aforesaid system comprising a mechanism as described above and means of interaction between the aforesaid light source and the skin zone to be treated, the aforesaid means of interaction being equipped to cooperate with the aforesaid means of control.

Finally the purpose of the invention is a dermatological treatment process implementing a device or a system as described previously.

### DESCRIPTION OF THE FIGURES

FIG. 1, already described, illustrates one of the difficulties that needs to be resolved in the context of this invention.

FIG. 2, already described, illustrates another difficulty that needs to be resolved in the context of this invention.

FIGS. 3 and 4, already described, illustrate a third difficulty that needs to be resolved in the context of this invention.

FIG. 5 is a diagrammatic representation of a device according to the invention.

FIG. 6 is a diagrammatic representation of an infrared sensor used in the context of this invention.

FIG. 7 is a diagrammatic representation of an initial method of producing the invention.

FIG. 8 is a diagrammatic representation of a second method of producing the invention.

FIG. 9 is a diagrammatic representation of a third method of producing the invention.

FIG. 10 is a diagrammatic representation of a variation of the method of producing the invention shown in FIG. 9.

FIG. 11 is a representation of the changes over a period of time of two temperatures that occur in the context of the illustration in FIG. 1.

FIG. 12 is a representation of the changes over a period of time of two temperatures that occur in the context of the illustration in FIG. 2.

FIG. 13 is a representation of the changes over a period of time of two temperatures that occur in the context of the illustration in FIGS. 3 and 4.

FIG. 14 is a representation of the changes over a period of time of two temperatures that occur in the context of the method of producing the invention in which the means of control are of the on/off type.

FIG. 15 is a representation of the changes over a period of time of two temperatures that occur in the context of the method of producing the invention in which the means of control are of the regulation type.

FIG. 16 is a diagrammatic representation of a dermatological treatment system comprising a device according to the invention; and

FIG. 17 is a diagrammatic representation of the field of view of an infrared sensor of a skin zone treated in the presence of an objective lens.

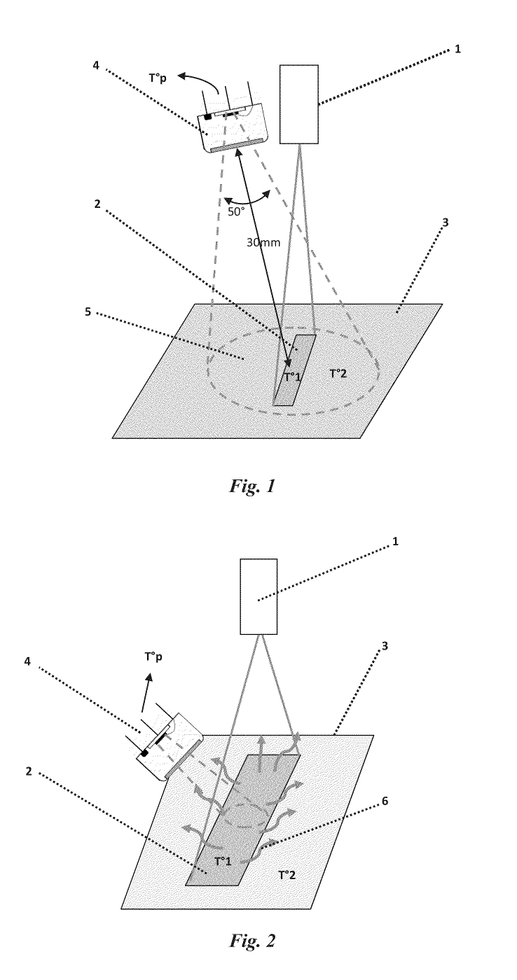

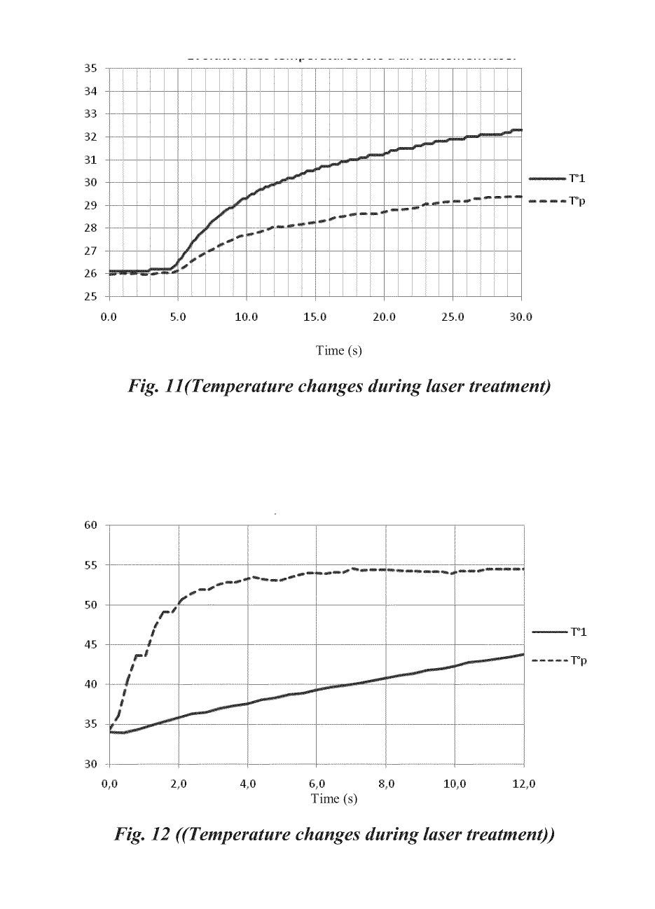

It should be noted that in FIGS. 5 to 17 the same reference figures have been used as those used in FIGS. 1 to 4 for equivalent elements.

### DETAILED DESCRIPTION OF THE INVENTION

In respect of the light source, it is possible to use a laser source or an LED source, but preferably a laser source. The aforesaid laser source emits within the wavelength from 0.6 μm to 2.5 μm, preferably from 0.7 to 2.2 μm and particularly preferably from 1.1 to 1.6 μm such as 1.21 μm. This light source permits the delivery, on the skin surface being treated, of a fluence between 1 and 500 J/cm² of skin, preferably a fluence between 2 and 250 J/cm² and particularly preferably a fluence between 5 and 200 J/cm².

In the context of the device according to the invention, the field of view of the infrared sensor is not therefore more divergent but has a contraction zone (focal point) around which the diameter of the field of view is minimal. One can thus in practice obtain fields of view with a diameter between 1.5 mm and 8 mm at a distance from the sensor of between 10 mm and 60 mm. It is thus possible to distance the sensor from the treatment area and thus avoid disturbance due to diffusion of the laser. In fact, the intensity of light disturbance diminishes proportionally to the square of the distance between the sensor and the treatment zone.

This arrangement is also favourable with regard to the first difficulty mentioned above, that is a sensor field of view greater than the treatment zone which also considerably falsifies the infrared sensor's measurements.

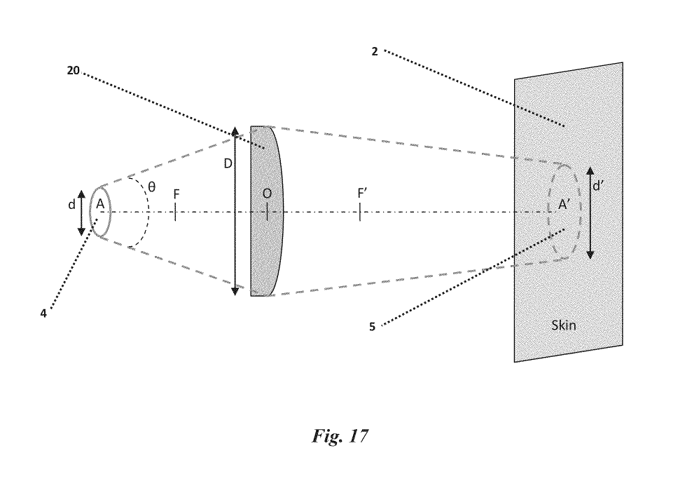

Concerning more specifically the objective lens (20), FIG. 17 represents the different parameters that need to be incorporated when choosing and positioning this lens in relation to the infrared sensor (4) and the area of skin to be treated (2) such that the skin area contained in the infrared sensor's field of view (5) is fully included in the zone of skin (2) treated by the dermatological treatment device.

More specifically, in this figure:
A and d respectively represent the centre and the diameter of the photosensitive element of the infrared sensor (4). e represents the angle of the field of view of the photosensitive element in the absence of the objective lens (2).
A′ and d′ respectively represent the centre and the diameter of the image (5) of the photosensitive element on the patient's skin, the image being fully included in the skin area (2) being treated by the light beam.
F is the primary focal point of the lens, F′ is the secondary focal point of the lens, O is the centre of the lens, and D is the diameter of the objective lens.

More specifically, the inventors have been able to demostrate that the position and diameter of the objective lens (20) can be determined by solving the following three equations:

$$\begin{matrix}\frac{1}{{\overline{OA}}^{′}} - \frac{1}{\overline{OA}} = \frac{1}{{f}^{′}} , & 1 ) \\ \frac{\overline{{d}^{′}}}{\overline{d}} = \frac{\overline{{OA}^{′}}}{\overline{OA}} , and & 2 ) \\ D = OA * Tan ( θ ) + d , & 3 )\end{matrix}$$

where
OA is the distance between the centre of the photosensitive element of the infrared sensor and the centre of the objective lens with OA being between 0 and 100 mm, preferably between 5 and 50 mm, and particularly preferably between 10 and 30 mm.
OA′ is the distance between the centre of the objective lens and the surface of the skin to be treated with:
   i) the sum of OA′ and OA being greater than or equal to 10 mm, preferably greater than or equal to 30 mm, and particularly preferably greater than or equal to 50 mm, and
   ii) the sum of OA′ and OA being less than or equal to 500 mm, preferably less than or equal to 100 mm, and particularly preferably less than or equal to 75 mm.
d is the diameter of the photosensitive element of the infrared sensor with d being between 0.1 and 10 mm, preferably between 0.5 and 5 mm, and particularly preferably between 1 and 3 mm.
θ represents the angle of the field of view of the photosensitive element of the infrared sensor in the absence of the objective lens with 8 being between 10 and 80°, preferably between 15 and 60°, and particularly preferably between 20 and 40°.
d′ is the diameter of the image of the photosensitive element on the patient's skin, the image needing to be fully included in the skin surface to be treated by the dermatological treatment device with:
   i) d′ being less than 20 mm, preferably less than 10 mm, and particularly preferably less than 5 mm, and
   ii) being greater than 0.5 mm, preferably greater than 1 mm.
f′ is the focal length of the objective lens.
D is the diameter of the objective lens with D being between 3 and 100 mm, preferably between 4 and 50 mm, and particularly preferably between 5 and 15 mm.

The diameter and position determined for the objective lens mean that the field of view of the aforesaid infrared sensor can be focused such that it is fully included in the skin area to be treated.

The inventors have thus been able to demonstrate that a device incorporating an infrared sensor and an objective lens (20), having the specific features described previously, enable a patient to be treated effectively (temperature greater than 45° C.) and burns to be avoided (temperature lower than 60° C.).

Advantageously, the device according to the invention could in addition include means of filtration composed of a material transparent in the wavelength range 6 μm to 10 μm in order to permit temperature measurement, the aforesaid material being opaque in the wavelength range from 0.6 μm to 2.5 μm. As an example of such materials, one could cite silicon or germanium, preferably silicon.

In this scenario, the disturbance of the diffusion of the light beam then becomes almost negligible due to the “selective wavelength filter”.

In some cases, the aforesaid means of filtration could correspond to the objective lens, in particular when the latter is made from silicon or germanium, preferably silicon.

In one particular method of producing the invention, the means of control is of the on/off type suitable for switching off the light source when the temperature measured by the infrared sensor in the skin zone being treated exceeds a predetermined value.

This method of producing the invention essentially aims at avoiding burns in the zone being treated by interrupting the operation of the light source when the temperature measured reaches a critical threshold. Treatment may eventually be resumed when the temperature drops below a second threshold.

In another particular method of producing the invention, the means of control are of the regulation type suitable for adjusting the power of the light source in order to maintain the temperature measured by the infrared sensor in the skin zone being treated between two predetermined values.

This means of producing the invention permits regulation of the power of the light source according to the temperature measured in order to maintain this latter temperature at an optimum value given the particular nature of the treatment to be applied and the characteristics of the skin being treated. It may be combined with the previous method of production as a safety measure in order to interrupt the operation of the light source if the temperature measured reaches a critical threshold.

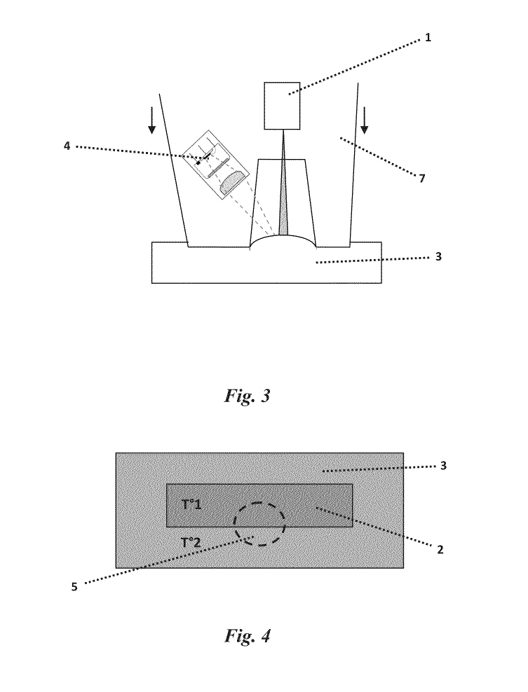

An additional difficulty results from the fact that the device is intended to be used on the skin of patients in the operating theatre, via a head (7) applied directly to the skin, as shown in FIG. 3). Skin is a flexible medium which deforms easily and the surface of which is rarely flat. In addition skin flexibility is variable from one zone of the body to another and from one individual to another (for example depending on age or corpulence).

Skin deformation also depends on the user, depending on the force with which the head of the device is applied to the patient.

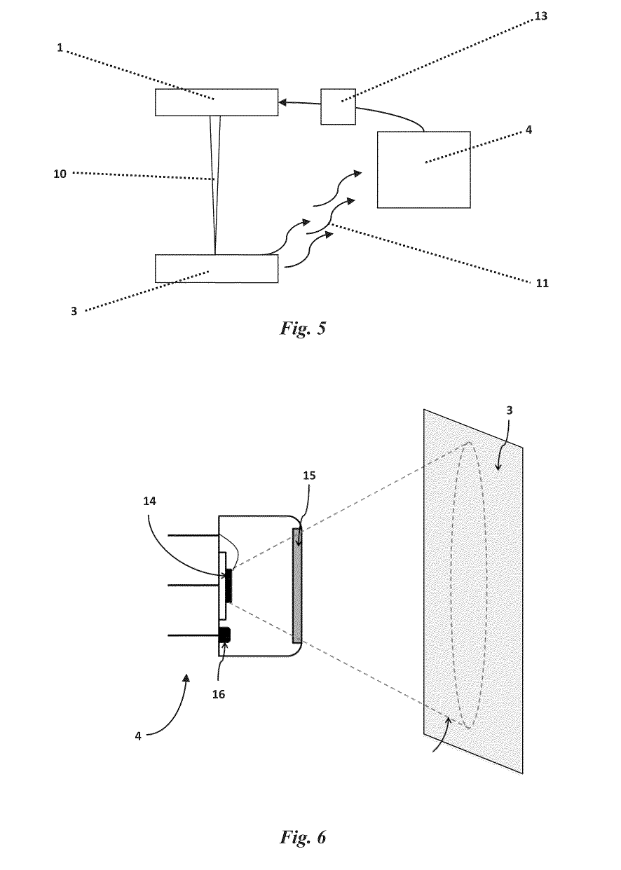

This deformation has three results, illustrated in FIG. 4:
The skin moves closer to the infrared sensor, reducing the sensor/treatment zone distance and therefore increasing the disturbance of the diffusion of the light beam.
The device's light beam is divergent, such that the dimensions of the beam increase with the distance it travels. When the skin deforms, the path travelled by the light beam is shorter; the area being treated is smaller and the light energy per surface unit is greater. The dose delivered per surface unit will not therefore conform to the dose scheduled by the device.
The skin is no longer positioned at the point of convergence of the light beam and the sensor's field of view (FIG. 4). The sensor's field of view (5) can no longer be fully contained within the zone (2) irradiated by the light beam. The temperature measurement will therefore be false.

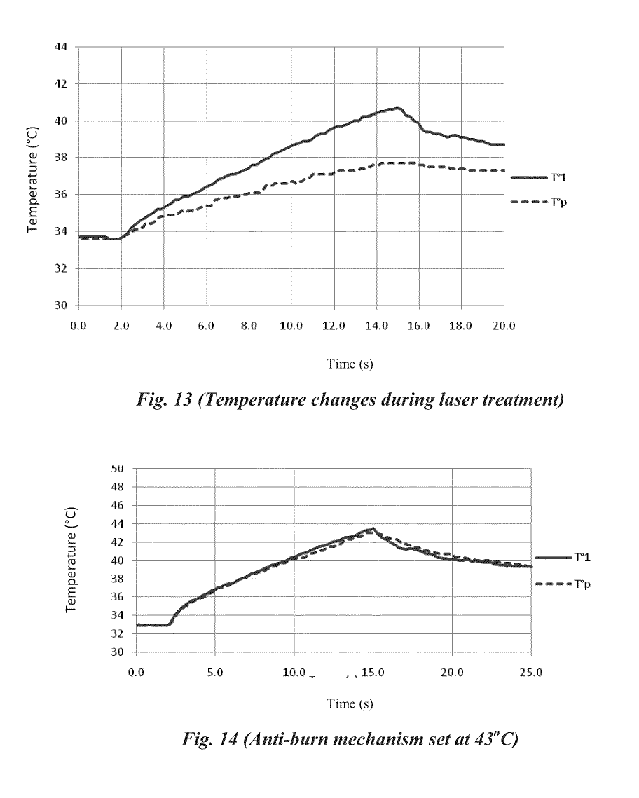

FIG. 13 shows the effect of the misalignment between the zone (2) being treated by the light beam and the infrared sensor (12) as shown in FIG. 4. The temperature measured is not that of the zone irradiated by the light beam:

T°₁>T°ₚ>T°₂

The invention also aims to mitigate this drawback and to this end has a mechanism as described previously comprising, in one particular method of producing the invention, a head that can be applied to a part of the skin comprising the area to be treated, in which the aforesaid head comprises a means of smoothing the surface to be treated.

More particularly, the aforesaid head may comprise a cavity equipped with an opening that can be applied to the surface of the skin to be treated, the light beam and the field of view of the means of measurement passing through the aforesaid cavity and arriving in the aforesaid opening, the aforesaid cavity being partially closed by an internal lip peripheral to the aforesaid opening, noticeably flat and which can be applied to the surface of the skin to be treated.

As a variation, the aforesaid opening can be closed with a window made from material that is transparent to the light beam and the radiation detected by the means of measurement.

The purpose of the invention is also as a system for dermatological treatment using a light beam, the aforesaid system comprising a mechanism as described previously and a means of interaction between the aforesaid light source and the skin surface to be treated, the aforesaid means of interaction being equipped to cooperate with the aforesaid means of control.

More particularly, the aforesaid means of interaction may comprise an adhesive medium equipped with means of identification and which can be fixed close to the skin zone to be treated, and an interface between the aforesaid adhesive medium and the means of control.

The invention permits a process of dermatological treatment to be implemented comprising stages consisting of:
Directing the light beam of a device as described previously onto the zone of the skin surface to be treated.
Measuring, using the means of measurement described previously, the temperature of the skin surface contained within the field of view of the aforesaid device's infrared sensor, the said skin surface being fully contained within the skin zone to be treated by the aforesaid device, and
To control the aforesaid light source via the aforesaid means of measurement such that the temperature of the skin zone to be treated is between 45 and 60° C.

We will now describe, by way of a non-exhaustive example, a method of producing the invention referring to the diagrammatic drawings in the appendix.

In FIG. 5, a device according to the invention can be seen comprising a light source (1) emitting a light beam (10) in a wavelength range of between 0.8 μm and 1.8 μm directed at part of a patient's skin (3) with a view to dermatological treatment.

Depending on its temperature, skin emits known infrared radiation (11) in a wavelength between 6 μm and 10 μm. This infrared heat radiation is detected by a sensor (4) of any known type. The output from the sensor is applied to the input of a regulator (13) which controls the source (1) in terms of power and/or exposure time.

FIG. 6 provides a more detailed view of the sensor (4).

The sensor (4) comprises a detector (14) sensitive to infrared heat (thermopile, pyro-electric). A selective wavelength filter (15) (transparent from 3 μm to 12 μm for example) is here added before the detector (14) in order to avoid disturbance by other wavelengths.

The signal returned by the detector (14) is a voltage V in the form:

V=α*ε*σ*(T_{obj}⁴−Tᵢₙₜₑᵣₙₐ⁴)

where T_{obj} is the temperature of the part (5) of the skin (3) placed in the field of view of the sensor, Tᵢₙₜₑᵣₙₐₗ is the internal temperature of the infrared sensor and a is a proportionality constant.

In order to deduce the skin temperature based on the signal from the infrared detector (14), it is necessary to know the sensor's internal temperature. In order to achieve this a temperature probe (16) is added as close as possible to the infrared detector (14) and the signal from the sensor is adjusted in compensation. If

Tᵢₙₜₑᵣₙₐ=T_{internamesurés}

then it is possible to deduce T_{obj}:

$${T}_{obj} = \sqrt[4]{\frac{V}{α * ɛ * σ} + {T}_{internemesur {e}^{'} s}^{4}}$$

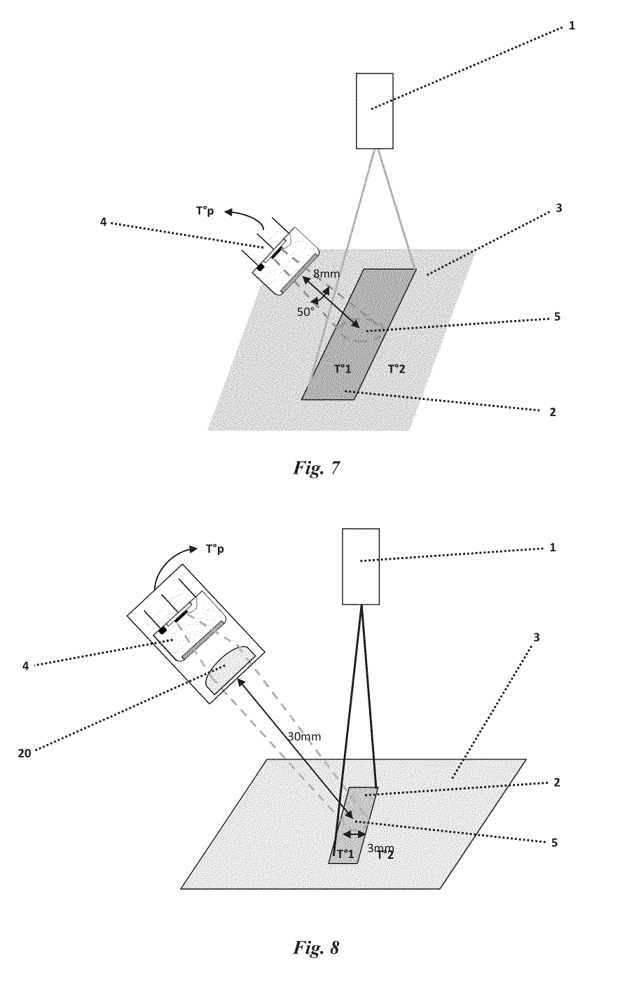

We will now refer to FIGS. 1 and 7.

We have previously described the problems posed by an arrangement of the type shown in FIG. 1.

In the case of FIG. 7, a sensor (4) is used, the divergence of the field of view of which is limited. By placing such a sensor as close as possible to the zone being treated (5) (between 5 mm and 10 mm), without for all that obstructing the passage of the light beam, the whole of the sensor's field of view (5) is contained in the zone irradiated by the light beam.

The area S₂ viewed by the infrared sensor when it is placed 8 mm from the irradiated zone is equal to:

$${S}_{z} = 0.8 * tan ( \frac{{50}^{°}}{2} ) = 0.44 {cm}^{2}$$

In the case of FIG. 7 where S₂ is equal to 0.44 cm² it is possible to measure the temperature of zones with an area of more than 0.5 cm²:

T°ₚ=T°₁

We will now refer to FIGS. 2 and 8.

In the method of production of the invention shown in FIG. 8, the sensor (4) has been moved to a position 30 mm from the treatment zone (2) in order to avoid disturbance resulting from the diffusion of the light beam. In order to resolve the problem of the sensor's field of view (5) which is greater than the treatment zone, a convergent lens (20) is used to focus the sensor's field of view.

For example, an infrared sensor (4) combined with an objective lens (20) gives a field of view 3 mm in diameter at a distance of 30 mm (that is a measurement area of 0.07 cm²), which permits the temperature measurement of zones with an area of over 0.1 cm². In the case of FIG. 8

T°ₚ=T°₁

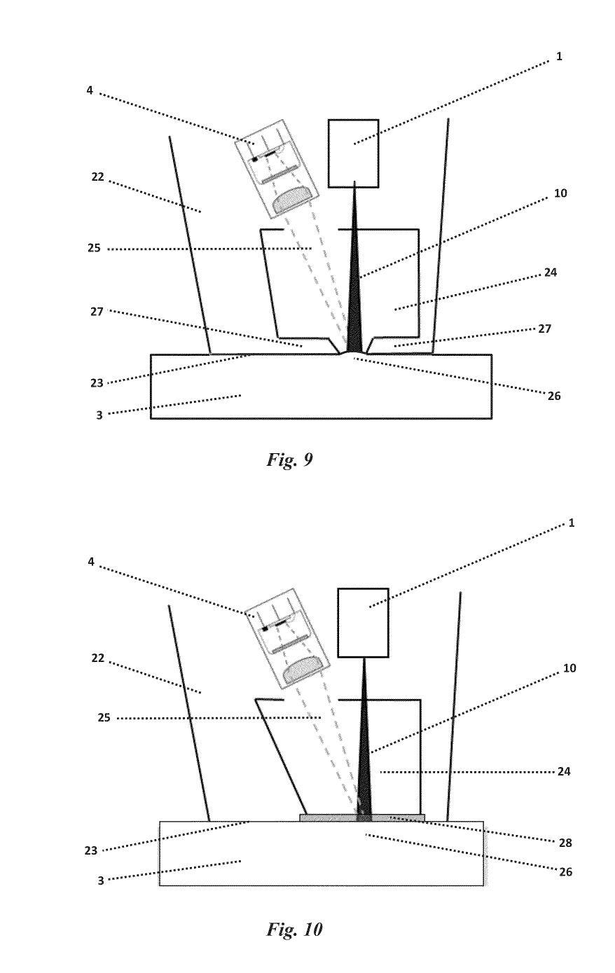

We will now refer to FIGS. 3, 4, 9 and 10. The purpose of the methods of production in FIGS. 9 and 10 is to avoid skin creasing likely to affect the temperature measurement.

Here the treatment is applied using a head (22) comprising the light source (1) and the infrared sensor (4). The head (22) includes a base (23) that comes into contact with the skin (3). The head (22) in addition forms a cavity (24) traversed by the beam (10) emitted from the light source (1) and by the field of view (25) of the infrared sensor (4). The cavity (24) has an opening (26) in the base (23), the beam (10) and the field of view (25) arriving in this opening.

In the method of producing the invention in FIG. 9, the base (23) forms a lip (27) surrounding and limiting the opening (26), and therefore partially closing the cavity (24). The opening (26) is of dimensions very slightly greater than those of the light beam.

The surface of the lip (27) external to the cavity (24) as a consequence limits the formation of skin creases. The more the opening is reduced, the more the skin is kept flat.

In the method of producing the invention in FIG. 10, the opening (26) is blocked by a window (28) made from material that is transparent to the light beam and the radiation detected by the sensor (4), that is to say it displays good optical transmission for the wavelengths of the light beam (0.6 μm to 2.5 μm) as well as in the thermal infrared range (6 μm to 10 μm), for example, in calcium fluoride. This window prevents the formation of skin creases. This latter mode of producing the invention is not really suitable however for use with the device when treating wounds because blood may dirty the window and directly absorb the light beam.

The invention permits the accurate measurement, in real time and without contact, of the temperature of a zone subject to homogeneous light radiation (wavelength range: 0.6 μm to 2.5 μm).

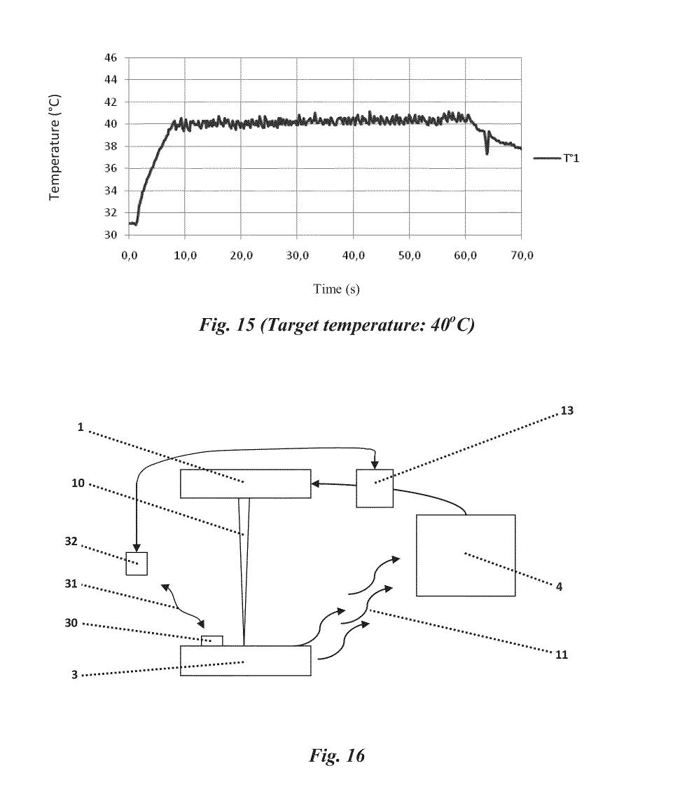

FIGS. 14 and 15 show the results obtained when:
The infrared sensor measurement area (4) is contained in the zone (2) irradiated by the light beam (area irradiated from 0.1 cm² to 2 cm²).
The distance infrared sensor/zone irradiated by the light beam is sufficiently large (from 10 mm to 60 mm) for the sensor (4) not to suffer from the effects of diffusion of the light beam by the skin.
The skin is now flat (anti-creasing) and is at the point of convergence of the light beam and the sensor's field of view (4).

FIG. 14 illustrates an initial mode of operation in which the means used to measure temperature is used as a safety device to prevent burning: when the temperature exceeds a certain predetermined threshold (for example between 40° C. and 70° C.) the light treatment is interrupted (43° C. in the figure).

FIG. 15 illustrates a second mode of operation in which the means used to measure temperature is used to control the temperature of the irradiated zone dynamically: the device adjusts its power and exposure time parameters to reach a predetermined temperature range (between 40° C. and 70° C.). Once the predetermined range has been reached, the device is also able to maintain the temperature over time. The temperature chosen is 40° C.; once reached, this temperature is maintained for 53 seconds.

FIG. 16 represents a dermatological treatment system comprising a device of the type described above.

This system comprises, apart from the elements of the device described above, a means of interaction between the light source (1) and the surface of the skin to be treated (3), arranged to cooperate with the control mechanism.

This means of interaction here comprises an adhesive medium (30) equipped with a means of identification, communicating via radio frequencies (31) with an interface (32) connected to the control mechanism (13).

Such a means of interaction is known through document WO 2008/107563 and will therefore not be described here in more detail.

1. A device for dermatological treatment using a laser beam, comprising:
a laser source (1) suitable for directing a light beam onto at least a zone (2) of the surface of the skin to be treated.
a means of contactless measurement sensitive to radiation according to temperature, of the temperature of a skin surface corresponding to the skin zone (2) treated, and
a means of controlling (13) the aforesaid laser source via the aforesaid means of measurement;
the aforesaid device being characterised in that the aforesaid means of contactless measurement comprises an infrared sensor (4) and an objective lens (20) suitable for focusing the field of view (5) of the aforesaid infrared sensor such that the skin surface contained within the aforesaid field of view (5) is wholly included in the area of skin (2) treated by the aforesaid device.

2. The device according to claim 1 wherein the wavelength of the laser source is from 1.1 to 1.6 μm such as 1.21 μm, such a wavelength enable to obtain a homogenous heating at a depth of at least 2 mm.

3. The device according to claim 1 characterised by the fact that the position and diameter of the objective lens (20) are determined by solving the following three equations:
$$\begin{matrix}\frac{1}{{\overline{OA}}^{′}} - \frac{1}{\overline{OA}} = \frac{1}{{f}^{′}} , & 1 ) \\ \frac{\overline{{d}^{′}}}{\overline{d}} = \frac{\overline{{OA}^{′}}}{\overline{OA}} , and & 2 ) \\ D = OA * Tan ( θ ) + d & 3 )\end{matrix}$$
where
OA is the distance between the centre of the photosensitive element of the infrared sensor (4) and the centre of the objective lens (20) with OA being between 0 and 100 mm, preferably between 5 and 50 mm, and particularly preferably between 10 and 30 mm
OA′ is the distance between the centre of the objective lens (20) and the surface of the skin treated (2) with:
i) the sum of OA′ and OA being greater than or equal to 10 mm, preferably greater than or equal to 30 mm, and particularly preferably greater than or equal to 50 mm, and
ii) the sum of OA′ and OA being less than or equal to 500 mm, preferably less than or equal to 100 mm, and particularly preferably less than or equal to 75 mm
d is the diameter of the photosensitive element of the infrared sensor (4) with d being between 0.1 and 10 mm, preferably between 0.5 and 5 mm, and particularly preferably between 1 and 3 mm
θ represents the angle of the field of view (5) of the photosensitive element of the infrared sensor (4) in the absence of the objective lens (20) with θ being between 10 and 80°, preferably between 15 and 60°, and particularly preferably between 20 and 40°
d′ is the diameter of the image of the photosensitive element on the patient's skin, the image needing to be fully included in the skin surface to be treated (2) by the dermatological treatment device with:
i) d′ being less than 20 mm, preferably less than 10 mm, and particularly preferably less than 5 mm, and
ii) d′ being greater than 0.5 mm, preferably greater than 1 mm
f′ is the focal length of the objective lens (20).
D is the diameter of the objective lens (20) with D being between 3 and 100 mm, preferably between 4 and 50 mm, and particularly preferably between 5 and 15 mm;
the diameter and position determined for the objective lens (20) mean that the field of view (5) of the aforesaid infrared sensor (4) can be focused such that it is fully included in the skin zone (2) being treated.

4. The device according to claim 1, characterised by the fact that the aforesaid means of control (13) is of the on/off type suitable for switching off the laser source (1) when the temperature measured by the infrared sensor (4) on the zone (2) of skin being treated exceeds a predetermined value.

5. The device according to claim 1, characterised by the fact that the aforesaid means of control (13) is of the regulation type suitable for adjusting the power of the laser source (1) in order to maintain the temperature measured by the infrared sensor (4) on the zone (2) of skin being treated between two predetermined values.

6. The device according to claim 1, characterised by the fact that in addition it comprises a means of filtration arranged between the infrared sensor and the surface of the skin being treated and composed of a material transparent in the wavelength range from 6 μm to 10 μm in order to permit temperature measurement, and opaque in the wavelength range from 0.6 μm to 2.5 μm.

7. The device according to claim 6, characterised by the fact that the aforesaid means of filtration are made from silicon or germanium, preferably silicon.

8. The device according to claim 1, characterised by the fact that it in addition comprises a head (22) that can be applied to a part of the skin comprising the surface to be treated, in which the aforesaid head includes a means of smoothing (27; 28) the surface being treated.

9. The device according to claim 8, characterised by the fact that the aforesaid head comprises a cavity (24) equipped with an opening (26) that can be applied to the surface of the skin to be treated, the light beam and the field of view of the means of measurement passing through the aforesaid cavity and arriving in the aforesaid opening, the aforesaid cavity being partially closed by an internal lip (27) peripheral to the aforesaid opening, noticeably flat and which can be applied to the surface of the skin to be treated.

10. The device according to claim 8, the aforesaid head comprising a cavity equipped with an opening that can be applied to the surface of the skin to be treated, the light beam and the field of view of the measuring device passing through the aforesaid cavity and arriving in the aforesaid opening, the aforesaid opening being closed by a window (28) made from material that is transparent to the light beam and the radiation detected by the means of measurement.

11. A system of dermatological treatment using a laser beam, characterised by the fact that it comprises a device according to claim 1 and a means of interaction (30, 32) between the aforesaid laser source and the area of skin being treated, the aforesaid means of interaction being arranged to cooperate with the aforesaid means of control (13).

12. The device according to claim 2 characterized by the fact that the position and diameter of the objective lens (20) are determined by solving the following three equations:
$$\begin{matrix}\frac{1}{{\overline{OA}}^{′}} - \frac{1}{\overline{OA}} = \frac{1}{{f}^{′}} , & 1 ) \\ \frac{\overline{{d}^{′}}}{\overline{d}} = \frac{\overline{{OA}^{′}}}{\overline{OA}} , and & 2 ) \\ D = OA * Tan ( θ ) + d & 3 )\end{matrix}$$
where
OA is the distance between the centre of the photosensitive element of the infrared sensor (4) and the centre of the objective lens (20) with OA being between 0 and 100 mm, preferably between 5 and 50 mm, and particularly preferably between 10 and 30 mm
OA′ is the distance between the centre of the objective lens (20) and the surface of the skin treated (2) with:
i) the sum of OA′ and OA being greater than or equal to 10 mm, preferably greater than or equal to 30 mm, and particularly preferably greater than or equal to 50 mm, and
ii) the sum of OA′ and OA being less than or equal to 500 mm, preferably less than or equal to 100 mm, and particularly preferably less than or equal to 75 mm
d is the diameter of the photosensitive element of the infrared sensor (4) with d being between 0.1 and 10 mm, preferably between 0.5 and 5 mm, and particularly preferably between 1 and 3 mm
θ represents the angle of the field of view (5) of the photosensitive element of the infrared sensor (4) in the absence of the objective lens (20) with θ being between 10 and 80°, preferably between 15 and 60°, and particularly preferably between 20 and 40°
d′ is the diameter of the image of the photosensitive element on the patient's skin, the image needing to be fully included in the skin surface to be treated (2) by the dermatological treatment device with:
i) d′ being less than 20 mm, preferably less than 10 mm, and particularly preferably less than 5 mm, and
ii) d′ being greater than 0.5 mm, preferably greater than 1 mm
f′ is the focal length of the objective lens (20).
D is the diameter of the objective lens (20) with D being between 3 and 100 mm, preferably between 4 and 50 mm, and particularly preferably between 5 and 15 mm;
the diameter and position determined for the objective lens (20) mean that the field of view (5) of the aforesaid infrared sensor (4) can be focused such that it is fully included in the skin zone (2) being treated.

13. The device according to claim 9, the aforesaid head comprising a cavity equipped with an opening that can be applied to the surface of the skin to be treated, the light beam and the field of view of the measuring device passing through the aforesaid cavity and arriving in the aforesaid opening, the aforesaid opening being closed by a window (28) made from material that is transparent to the light beam and the radiation detected by the means of measurement.
